(19) United States
(12) Patent Application Publication
(10) Pub. No.: US 2018/0207146 A1
(43) Pub. Date: Jul. 26, 2018

(54) PHARMACEUTICAL COMPOSITION COMPRISING 5ALPHA-REDUCTASE INHIBITOR

(71) Applicant: CHONG KUN DANG PHARMACEUTICAL CORP., Seoul (KR)

(72) Inventors: Byoung Ki AHN, Gyeonggi-do (KR); Ki Seong KO, Gyeonggi-do (KR); So Hyun PARK, Gyeonggi-do (KR); Min Hyo KI, Gyeonggi-do (KR); Jong Lae LIM, Gyeonggi-do (KR)

(21) Appl. No.: 15/327,247

(22) PCT Filed: Jul. 20, 2015
(86) PCT No.: PCT/KR2015/007513

§ 371 Date: Jan. 18, 2017

(30) Foreign Application Priority Data
Jul. 21, 2014 (KR) 10-2014-0091774

(51) Int. Cl. A61K 31/473; A61K 31/542; A61K 9/08; A61K 31/5415; A61K 31/405; A61K 31/407; A61K 9/00; A61K 47/14; A61K 47/24; A61K 47/28; A61K 47/22

(52) U.S. Cl. CPC A61K 31/473; A61K 31/542; A61K 9/08; A61K 31/5415; A61K 31/405; A61K 47/22; A61K 9/0019; A61K 47/14; A61K 47/24; A61K 47/28; A61K 31/407

(57) ABSTRACT

Disclosed is a composition comprising a 5α-reductase inhibitor. Forming a liquid crystal upon exposure to an aqueous fluid, the composition can release the 5α-reductase inhibitor at a constant rate over a long period of time. In addition, the composition can significantly alleviate irritations attributed to the topical administration of the 5α-reductase inhibitor, and thus the composition has improved safety.

### TECHNICAL FIELD

The present invention relates to a composition comprising a 5α-reductase inhibitor as a pharmaceutically active substance.

### BACKGROUND ART

Sustained release formulations are designed to consistently release a pharmacologically active ingredient at a single dose in order to maintain effective plasma concentration of the substance in the blood stream for a specific period of time, with minimization of the side effects caused by multiple doses.

A liquid crystal is a technique associated with sustained release formulations. The liquid crystal having the non-lamellar and bi-continuous structure is adapted to apply to a sustained-release injection drug delivery system. At a site to which a liquid crystal formulation containing a drug is injected intramuscularly or subcutaneously, a gel-type liquid crystal is formed and the drug is released at a constant rate over several months, thus playing a role as a sustained-release injection. Therefore, the liquid crystal technique allows for the provision of a pharmaceutical composition that increases medication compliance and last the pharmaceutical effect for a long period of time.

5α-reductase is an enzyme that catalyzes the conversion of testosterone, an androgen, into dihydrotestosterone (DHT) in the prostate, hair follicles, sebaceous glands, etc. Dihydrotestosterone is known to associate with alopecia and benign prostatic hyperplasia, etc. A 5α-reductase inhibitor, which inhibits the production of dihydrotestosterone in tissues, is used as a therapeutic agent for benign prostatic hyperplasia, and in the prevention and treatment of alopecia. Benign prostatic hyperplasia (BPH) is a senile disease in which the urethra is compressed to cause dysuria. The causes of benign prostatic hyperplasia are accounted for by aging and the androgen. Dihydrotestosterone converted from testosterone by 5α reductase in prostate gland elicits the development and enlargement of the prostate gland, causing the onset of benign prostatic hyperplasia. This disease can be treated by the excision of prostatic tissues. However, preference is made for a medicine therapy not only because surgical operations may be limited for elderly patients, but also because there is post-operative side effects and recurrence. By selectively inhibiting the production of dihydrotestosterone, 5α-reductase inhibitors exhibit anti-androgen effects and reduce an enlarged prostate gland, thus improving dysuria.

Alopecia is a condition in which the rate of hair loss is larger than that of hair regrowth, and has various causes including a genetic background, nutritional deficiency, stress, etc. Androgenic alopecia, the most common hair loss, is associated with excessive dihydrotestosterone production. In detail, dihydrotestosterone suppresses the growth of hair follicular cells by lowering energy generation and protein synthesis around hair follicles while inducing the formation of proteins responsible for alopecia to trigger androgenic alopecia. In addition, a tissue where hair loss is ongoing has higher 5α-reductase activity than other scalp tissues. Hence, 5α-reductase inhibitors are applicable to the effective prevention and so treatment of androgenic alopecia by suppressing dihydrotestosterone activity.

Commercially available 5α-reductase inhibitors are now finasteride (Propecia®, Proscar®), dutasteride (Avodart®). etc. Finasteride (Propecia®, Proscar®) is a type II 5α-reductase inhibitor and is administered once a day at 5 mg for benign prostatic hyperplasia and 1 mg for alopecia. Dutasteride (Avodart®) inhibits type I and II 5α-reductase, and is administered once a day at 0.5 mg for both benign prostatic hyperplasia and alopecia. 5α-Reductase inhibitors are regarded as relatively safe therapeutic agents because they have no effects on the androgen testosterone, but selectively inhibit the production of dihydrotestosterone. For the therapy of benign prostatic hyperplasia or alopecia, in addition, the drugs need to be administered for a long period of time or for the life of the patient. So, the commercially available drugs are poor medication compliance even though they are designed to be administered once a day.

Based on previous inventions, the present inventors provide a pharmaceutical composition comprising a 5α-reductase inhibitor as a pharmaceutically active agent, which exists as a liquid phase in the absence of an aqueous fluid, and forms a liquid crystal upon exposure to the aqueous fluid. The pharmaceutical composition is designed to sustained-release the pharmaceutically active substance 5α-reductase inhibitor, thereby improving safety and medication compliance.

Previously, the present inventors invented a sustained-release lipid pre-concentrate using a liquid crystal technique, and a pharmaceutical composition comprising the same, as described in Korean Patent Application Nos. KR10-2012-0093677, KR10-2012-0157562, KR10-2012-0157582, and KR10-2012-0157583.

However, the injection of the 5α-reductase inhibitors in such a pre-concentrate composition is likely to cause severe irritation at the injection site. Because safety should be taken into account for product development and clinical application, there is therefore a need for a novel formulation that overcomes the problem of severe irritation upon topical injection of the 5α-reductase inhibitors while achieving sustained release through the liquid crystal formation.

Hereinafter, reference is now made to prior arts relevant to the present invention.

International Patent Publication No. WO 1996/012817 discloses a combination method using pharmaceutically active substances useful in the treatment of acne. Briefly, 5α-reductase inhibitors and anti-inflammatory agents are co-administered for the treatment of acne. The invention of this patent application is irrelevant to the liquid crystal, and different from the present invention in that it is concerned with the treatment of acne.

International Patent Publication No. WO 2004/006937 describes a non-biodegradable polymer-based, sustained-release composition useful as a subcutaneous implant composition. Also, the 5α-reductase inhibitors, especially finasteride or dutasteride can be used as an active ingredient in the subcutaneous implant composition. However, this patent application is different from the present invention in that the composition cannot form the liquid crystal, and employs the non-biodegradable polymer.

International Patent Publication No. WO 2013/142274 discloses a method for reducing the urinary frequency, comprising co-administering an analgesic agent and the 5α-reductase inhibitor. However, this patent application is different from the present invention in that it is not associated with the liquid crystal, and is concerned with the treatment of urinary frequency.

U.S. Pat. No. 6,486,204 discloses a method for the treatment or prevention of prostate cancer by administering rofecoxib, a non-steroidal anti-inflammatory drug selectively inhibiting COX-2 , alone or in combination with other drugs, such as the 5α-reductase inhibitors. The patent, however, does not mention any composition forming the liquid crystal and relates to the use of rofecoxib as an anticancer drug, so that it is different from the present invention.

U.S. patent publication application No. 2006/0204588 discloses a nanoparticule formulation in an injectable depot dosage form, having a particle size of less than 2,000 nm. Finasteride, dutasteride, or tamsulosin may be employed as the pharmaceutically active agent and formulated together with phospholipids and polyoxyethylene sorbitan monoleate as stabilizers. However, the composition of this no patent application cannot form the liquid crystal and includes the form of particles with a size of less than 2,000 nm and polyoxyethylene sorbitan monooleate is a macromolecule in which polyethylene oxide is crosslinked. Therefore, this U.S. patent is different from the present invention, which does not form particles.

U.S. publication application No. 2013/0251786 discloses a lipid formation including a hyaluronan-degrading enzyme for the treatment of benign prostatic hyperplasia and also introduces the 5α-reductase inhibitor as a co-administrable therapeutic agent. In addition, this patent application discloses phosphatidylcholine is used as an amphipathic lipid involved in the liposome. However, the composition of this patent application does not form the liquid crystal, and the hyaluronan-degrading enzyme that is administered together with the 5α-reductase inhibitor is not employed in the present invention. Hence, this patent application is different from the present invention.

### RELATED ART DOCUMENT

[Patent Document]

(Patent Document 1) Korean Patent Application No. 10-2012-0093677

(Patent Document 2) Korean Patent Application No. 10-2012-0157562

(Patent Document 3) Korean Patent Application No. 10-2012-0157582

(Patent Document 4) Korean Patent Application No. 10-2012-0157583

(Patent Document 5) International Patent Publication No. WO 1996/012817

(Patent Document 6) International Patent Publication No. WO 2004/006937

(Patent Document 7) International Patent Publication No. WO 2013/142274

(Patent Document 8) U.S. Pat. No. 6,486,204

(Patent Document 9) U.S. Patent Publication No. US 2006/0204588

(Patent Document 10) U.S. Patent Publication No. US 2013/0251786

### DISCLOSURE

### Technical Problem

It is therefore an object of the present invention to provide a pharmaceutical composition including a 5α-reductase inhibitor which is sustained-released to improve a medication compliance of patients and prolong pharmaceutical effects.

It is another object of the present invention to provide a pharmaceutical composition that overcomes problems associated with the topical administration of a 5α-reductase inhibitor.

### Technical Solution

The present invention provides a composition comprising:

a 5α-reductase inhibitor; and

a non-steroidal anti-inflammatory drug (NSAID), corticosteroid, or a mixture iso thereof,

wherein the composition exists as a lipid phase in the absence of an aqueous fluid and forms into a liquid crystal upon exposure to the aqueous fluid.

The present invention administers the 5α-reductase inhibitor in combination with the non-steroidal anti-inflammatory drug, corticosteroid, or a mixture thereof to improve safety with a significant reduction in local irritation, such as erythema, inflammation, etc., occurring at an injection site of the 5α-reductase inhibitor.

In addition, the composition according to the present invention exists as a lipid liquid phase in the absence of the aqueous fluid, but is converted into the liquid crystal upon exposure to the aqueous fluid, such as upon injection into the body, and thus has high safety. After being injected into the body, the composition forms the liquid crystal. As a result, the 5α-reductase inhibitor, and the non-steroidal anti-inflammatory drug and/or corticosteroid are released slowly from the liquid crystal for one week or longer, and preferably for one month or longer, thereby increasing the medication compliance of patients, and duration of the pharmaceutical efficacy.

The 5α-reductase inhibitor interrupts conversion of testosterone into dihydrotestosterone (DHT), which is responsible for prostate enlargement, to reduce the differentiation and size of the prostate tissue, thereby increasing urine flow rate and alleviating dysuria. Having such an interruptive effect on dihydrotestosterone, the 5α-reductase inhibitors are used in the treatment of benign prostatic hyperplasia, androgenic alopecia and hypertrichosis, and as a therapeutic aid after radical prostatectomy. However, although their therapeutic effects require administration for several or more months, their formulations are commercially available for one daily dose of oral medication.

The 5α-reductase inhibitor available as the pharmaceutically active ingredient of the present invention may be selected from the group consisting of dutasteride, finasteride, bexlosteride, epristeride, izonsteride, lapisteride, turosteride, a pharmaceutically acceptable salt thereof, and a combination thereof. Preferably, the 5α-reductase inhibitor may be selected from the group consisting of dutasteride, finasteride, a pharmaceutically acceptable salt thereof, and a combination thereof. More preferably, the 5α-reductase inhibitor may be dutasteride or a pharmaceutically acceptable salt thereof, but without limitations thereto.

The non-steroidal anti-inflammatory drugs are a class of drugs that provide analgesic, antipyretic and anti-inflammatory effects, without a steroidal structure. Their therapeutic effects result from inhibiting the activity of COX (cyclooxygenase) enzyme, and thereby, the synthesis of prostaglandin which is mainly responsible for inflammation. In accordance with the present invention, the non-steroidal anti-inflammatory drug may be selected from the group consisting of include aceclofenac, acemetacin, alminoprofen, amfenac, apazone, aspirin, bromfenac, bufexamac, celecoxib, choline salicylate, cinnoxicam, clonixin, dexibuprofen, dexketoprofen, diclofenac, diflunisal, emorfazone, etodolac, etoricoxib, ethenzamide, felbinac, fenoprofen, flufenamic acid, flurbiprofen, ibuprofen, imidazole salicylate, indomethacin, isopropylantipyrine, ketoprofen, ketorolac, lornoxicam, loxoprofen, meclofenamate, meloxicam, mefenamic acid, morniflumate, nabumetone, naproxen, nefopam, nimesulide, oxaprozin, oxyphenbutazone, pelubiprofen, phenylbutazone, piroxicam, pranoprofen, proglumetacin, rofecoxib, salsalate, salicylate, sulindac, talniflumate, tenoxicam, tiaprofenic acid, tolfenamic acid, tolmetin, valdecoxib, zaltoprofen, a pharmaceutically acceptable salt thereof, and a combination thereof. Preferably, the non-steroidal anti-inflammatory drug may be selected from the group consisting of dexketoprofen, ibuprofen, indomethacin, ketoprofen, ketorolac, lornoxicam, meloxicam, nefopam, piroxicam, a pharmaceutically acceptable salt thereof and a combination thereof. More preferably, the non-steroidal anti-inflammatory drug may be selected from the group consisting of lornoxicam, meloxicam, a pharmaceutically acceptable salt thereof, and a combination thereof without limitations thereto.The corticosteroids is a drug having therapeutic effects such as anti-inflammation, immunosuppression, etc.

When corticosteroids bind to a glucocorticoid receptor, synthesis of lipocortin is promoted and the lipocortin inhibits activity of phopholipase-A2 to prohibit the biosynthesis of prostaglandin through blocking the arachidonate cascade. Thus, the corticosteroids elicit therapeutic effects such as anti-inflammation, immunosuppression, etc. In accordance with the present invention, the corticosteroid may be selected from the group consisting of beclomethasone dipropionate, betamethasone, budesonide, deflazacort, dexamethasone, difluprednate, epinephrine, fludrocortisone, fluocinolone acetonide, fluocortin, fluorometholone, fluticasone, hydrocortisone, methylprednisolone, prednisolone, prednisone, triamcinolone, a pharmaceutically acceptable salt thereof, and a combination thereof. Preferably, the corticosteroid may be selected from the group consisting of betamethasone, dexamethasone, epinephrine, hydrocortisone, methylprednisolone, prednisolone, triamcinolone, a pharmaceutically acceptable salt thereof, and a combination thereof. More preferably, the corticosteroid may be selected from the group consisting of betamethasone, dexamethasone, a pharmaceutically acceptable salt thereof, and a combination thereof without limitations thereto.

Because both the non-steroidal anti-inflammatory drug and the corticosteroid have anti-inflammatory and analgesic effects, both the non-steroidal anti-inflammatory drug and the corticosteroid can alleviate irritation at the injection site of the 5α-reductase inhibitor, thus improving the safety of the pharmaceutical composition.

The composition of the present invention exists as the liquid phase without the aqueous fluid such as water. However, when the composition of the present invention is exposed to the aqueous fluid such as water, the composition undergoes transition from the liquid phase to the liquid crystal of the semi-solid which is a gel state to release the active ingredients in a sustained manner. The composition of the present invention is different from conventional compositions that form a lamellar structure such as micelles, emulsions, microemulsions, liposomes and lipid bilayers, which have been widely used in designing the pharmaceutical formulations. Such lamellar structures are in oil in water (o/w) or water in oil (w/o) type in which there is clear discrimination inner and out phases.

In accordance with the present invention, the liquid crystals formed by the composition have a non-lamellar phase structure, preferably cubic or hexagonal, in which oil and water are in an ordered mixture and arranged without discrimination between inner and out phases. The ordered arrangement of oil and water renders the non-lamellar phase structure of a mesophase, which is a state of intermediate between the liquid phase and the solid phase. In addition, there are innumerous water paths, like Mobius strips, inside the non-lamellar liquid crystals, and the water paths may be surrounded with a lipid layer.

Because the composition of the present invention exists as the liquid state before administration to the human body, it can be readily administered through non-oral routes, for example, by injection and preferably by topical injection. After administered into the body, the composition of the present invention is transformed from the liquid phase into the non-lamellar liquid crystal phase exhibiting excellent sustained release of the drug within the body.

In accordance with an aspect thereof, the present invention provides a composition, comprising:

a) a sorbitan unsaturated fatty acid ester having two or more —OH (hydroxyl) groups in its polar head;

b) a phospholipid; c) a liquid crystal hardener that is free of ionizable groups and has a hydrophobic moiety of 15 to 40 carbon atoms comprising a bulky triacyl group or carbon ring structure; d) a 5α-reductase inhibitor as a pharmaceutically active agent; and e) a non-steroidal anti-inflammatory drug (NSAID), a corticosteroid, or a mixture thereof, wherein the composition exists as a liquid phase in the absence of an aqueous fluid, and forms a liquid crystal upon exposure to the aqueous fluid. In the composition of the present invention, the ingredients a) to c) are responsible for the formation of the liquid crystals in the body, thus allowing for the sustained release of the ingredients d) and e) within the body.

When the ingredients a) to c) of the composition of the present invention form into a liquid crystal upon exposure to the aqueous fluid, the active ingredients can be released at a constant rate in an excellent sustained manner for one or more weeks and preferably for one or more months. In addition, the composition of the present invention can prevent the active ingredients from a burst release in the early stage after the administration of the composition.

In the present invention, the sorbitan unsaturated fatty acid ester functions as a liquid crystal former. When used as the liquid crystal former in the composition of the present invention, the sorbitan unsaturated fatty acid ester can allow for the preparation of formulations with better sustained release properties, and can improve the safety of the composition due to its biodegradability compared to conventional liquid crystal formers. The sorbitan unsaturated fatty acid ester preferably has two or more -OH (hydroxyl) groups that may be directly attached to the heterocyclic alkyl ring of the polar head group in the sorbitan group. For example, the sorbitan unsaturated fatty acid ester may include tetrahydrofuran ring to which two or more -OH (hydroxyl) groups are directly bonded. The compound of Chemical Formula 1 is sorbitan monoester where R¹=R²=OH, R³=R, and sorbitan diester where R¹=OH, R²=R³=R, R being an alkyl ester of 4 to 30 carbon atoms with at least one unsaturated bond.

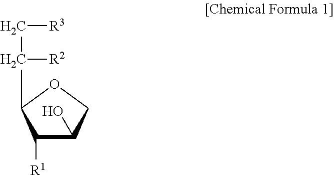

In detail, the sorbitan unsaturated fatty acid ester of the present invention is derived from the fatty acid ester obtained from whale oils and fish oils as well as vegetable oils (e.g., coconut oil, castor oil, olive oil, peanut oil, rapeseed oil, corn oil, sesame oil, cottonseed oil, soybean oil, sunflower seed oil, safflower seed oil, linseed oil, etc.) and animal fats and oils (e.g., milk fat, lard, beef tallow, etc.). The sorbitan unsaturated fatty acid ester of the present invention may be selected from the group consisting of sorbitan monoester, sorbitan sesquiester, sorbitan diester, and a combination thereof.

Sorbitan monoester is a compound in which one fatty acid group is ester-bonded to sorbitan, and may be selected from the group consisting of sorbitan monooleate, sorbitan monolinoleate, sorbitan monopalmitoleate, sorbitan monomyristoleate, and a combination thereof.

Sorbitan sesquiester is a compound in which 1.5 fatty acid groups, on average, are ester-bonded to sorbitan , and may be selected from the group consisting of sorbitan sesquioleate, sorbitan sesquilinoleate, sorbitan sesquipalmitoleate, sorbitan sesquimyristoleate, and a mixture thereof.

Sorbitan diester is a compound in which two fatty acid groups is ester-bonded to sorbitan and may be selected from the group consisting of sorbitan dioleate, sorbitan dilinoleate, sorbitan dipalmitoleate, sorbitan dimyristoleate, and a mixture thereof.

According to the present invention, the sorbitan unsaturated fatty acid ester may be preferably selected from the group consisting of sorbitan monooleate, sorbitan sesquioleate, sorbitan monolinoleate, sorbitan monopalmitoleate, sorbitan monomyristoleate, and a mixture thereof.

Phospholipids are essential for the construction of the lamellar phase structures, such as liposomes, in conventional techniques, but cannot form the non-lamellar phase structure, such as the liquid crystal, by themselves. However, the phospholipids can participate in the sorbitan unsaturated fatty acid ester-driven formation of the non-lamellar phase structures, serving to stabilize the resulting liquid crystals.

According to the present invention, the phospholipid is derived from plants or animals, and contains a saturated or unsaturated alkyl ester group of 4 to 30 carbon atoms. The phospholipid may be selected from the group consisting of phosphatidylcholine, phosphatidylethanolamine, phosphatidylserine, phosphatidylglycerine, phosphatidylinositol, phosphatidic acid, sphingomyelin, and a mixture thereof.

Also, the phospholipid may be derived from plants or animal products such as beans or eggs, and the alkyl ester groups of the phospholipid may include a saturated fatty acid chains such as mono- and dipalmitoyl, mono- and dimyristoyl, mono- and dilauryl, and mono- and distearyl, etc., and an unsaturated fatty acid chains such as mono- or dilinoleyl, mono- and dioleyl, mono- and dipalmitoleyl, and mono- and dimyristoleyl. In addition, phospholipids may include both saturated and unsaturated fatty acid esters.

The liquid crystal hardener of the present invention cannot form the non-lamellar structure, unlike the liquid crystal former, nor the lamellar structure unlike phospholipids, by itself. However, the liquid crystal hardener contributes to the liquid crystal former-driven formation of the non-lamellar phase structures by increasing the curvature of the non-lamellar phase structures to enhance the ordered co-existence of oil and water. In order to this function, the liquid crystal hardener is advantageously required to have a highly limited polar moiety and a bulky non-polar moiety inside its molecular structure.

In practice, however, biocompatible molecules which are injectable into the body can be selected as the liquid crystal hardener of the present invention only via direct and repeated experiments. As a result, the liquid crystal hardeners suitable for the composition of the present invention have molecular structures that are different from one another and thus cannot be elucidated as one molecular structure. The common structural feature deduced by observation of all of the liquid crystal hardeners identified is that they are free of ionizable groups, such as carboxyl and amine groups, and have hydrophobic moieties of 15 to 40 carbon atoms comprising a bulky triacyl group or a carbon ring structure.

Hence, examples of the liquid crystal hardener of the present invention may be selected from the group consisting of triglyceride, retinyl palmitate, tocopherol acetate, cholesterol, benzyl benzoate, ubiquinone and a mixture thereof without limitations thereto. Preferably, the liquid crystal hardener may be selected from the group consisting of tocopherol acetate, cholesterol, benzyl benzoate, and a mixture thereof.

The active ingredients d) and e) of the composition according to the present invention are as explained above.

According to the present invention, the composition exists as the lipid liquid phase in the absence of the aqueous fluid such as water, but when exposed to the aqueous fluid, the composition is converted into the liquid crystal to release the active ingredients in a sustained manner.

According to the present invention, the liquid crystals formed by the composition have the non-lamellar phase structure, preferably the cubic or hexagonal structure. In the non-lamellar liquid crystal of the present invention, oil and water are in an ordered mixture and arranged without discrimination between inner and out phases. The ordered arrangement of oil and water of the liquid crystal renders the non-lamellar phase structure of a mesophase, which is a state of the intermediate between liquid and solid. In addition, there are the innumerous water paths, like Mobius strips, inside the non-lamellar liquid crystals, and the water paths may be surrounded with a lipid layer.

Because the composition of the present invention contains the ingredients a) to c), the composition undergoes transition from the liquid phase to the non-lamellar liquid crystal phase upon exposure to the aqueous fluid and thus the active ingredients can be sustained-released at a constant rate for a long period of time. The composition employing the ingredients a) to c) can significantly prolong the release time of the active ingredient, reduce initial release burst, prohibit a prolonged lag time which is a time taken for the drug to be initially released and sustained-release the active ingredients at a constant rate for 30 days or longer from the administration of the composition compared to those employing conventional liquid crystal forming ingredients.

The composition of the present invention may further include an organic solvent for solubilization. The organic solvent may be preferably selected from the group consisting of N-methylpyrrolidone, dimethyl sulfoxide, benzyl alcohol, benzyl benzoate, dimethylacetamide, ethanol, and a combination thereof, without limitations thereto.

The composition of the present invention may be used in the prevention or treatment of benign prostatic hyperplasia, androgenic alopecia or hypertrichosis, or a therapeutic aid after radical prostatectomy.

The composition of the present invention may be parenterally administered. Parenteral administration may allow for improving the medicinal effect of the composition of the present invention compared to oral administration. Particularly, when the composition of the present invention is administered by the parenteral injection, irritation such as erythema, inflammation, etc., caused by the active ingredient 5α-reductase inhibitor can be reduced to significantly improve the symptom such as erythema, inflammation, etc.,.

The composition of the present invention may be topically administered. For example, the composition of the present invention may be applied in the form of an ointment or a gel to the skin or may be locally administered by injection.

The composition of the present invention may be injected via either a subcutaneous route or an intramuscular route. The injection route may be determined according to the properties of individual pharmaceutically active agents.

The composition of the present invention may be administered by injection to a local site. For example, when the composition of the present invention is used for the treatment of benign prostatic hyperplasia, the composition may be injected to a site around the prostate gland. In this case, the composition forms the liquid crystal phase in the hypodermis, and slowly releases the active ingredients included in the composition. When the composition of the present invention is applied to the treatment of alopecia, the composition may be injected into a predetermined scalp site that needs hair regrowth. In this case, the composition forms the liquid crystal phase in the hyperdermis of the scalp, and releases the active ingredients included in the composition in a sustained manner.

According to the present invention, the weight ratio of the ingredients a) and b) suitable for the formation of the liquid crystals desired by the pharmaceutical composition ranges from about 10:1 to about 1:50, and preferably from about 5:1 to about 1:10. The weight ratio of a)+b) and c) falls within the range of from about 1000:1 to about 1:1, and preferably from about 100:1 to about 2:1. Within the ranges, the desirable sustained release attributed to the liquid crystal of the present invention can be elicited more effectively. The sustained release profile can be controlled by adjusting the ratio of a), b) and c) in order to effectively prevent the initial release burst of drug, or lag time prolongation.

The weight ratio of a)+b)+c) and d) ranges from about 10000:1 to about 1:100, and preferably from about 1000:1 to about 1:10 in order to guarantee desirable sustained release of the pharmaceutically active ingredient 5α-reductase inhibitor in the pharmaceutical composition of the present invention.

In the pharmaceutical composition of the present invention, the weight ratio between a)+b)+c) and e) suitable for forming the liquid crystal is a range of about 10000:1 to about 1:1, and preferably in a range of about 1000:1 to about 5:1 in order to allows the non-steroidal anti-inflammatory drug for alleviating topical irritation.

Given these weight ranges of the ingredients a) to e), the pharmaceutical composition of the present invention efficiently sustained-release each of the active ingredient included in the composition.

As used herein, the term “aqueous fluid” is intended to include water and body fluids such as mucosal solution, tear, sweat, saliva, gastrointestinal fluid, extravascular fluid, extracellular fluid, interstitial fluid, and plasma. When the composition of the present invention is contacted with a surface of the body, a region or a cavities (for example, inside the body) in which the aqueous fluid exists, the composition is transformed from the liquid phase into the liquid crystal having a form of the semi-solid. That is, the composition of the present invention exists as the liquid state before application to the human body and is converted into the liquid crystal phase showing the sustained release within the body.

According to the present invention, under a very restricted condition, the liquid crystals formed by the composition of the present invention have the non-lamellar phase structure in which oil and water are in an ordered mixture and arranged without discrimination between inner and out phases. The ordered arrangement of oil and water renders the non-lamellar phase structure of the mesophase, which is a state of the intermediate between the liquid and the solid. The liquid crystal phase of the composition of the present invention is different the lamellar structures, such as micelles, emulsions, microemulsions, liposomes, and lipid bilayers, which have been widely used in a conventional pharmaceutical formulation design. Such lamellar structures are in oil in water (o/w) or water in oil (w/o) type in which there is clear discrimination of an inner phase and an out phase, and thus are different from the liquid crystals of the present invention.

According to the present invention, liquid crystallization in the ‘liquid crystals’ refers to the formation of the liquid crystals having the non-lamellar phase structure from the composition upon exposure to the aqueous fluid.

The pharmaceutically active ingredient 5α-reductase inhibitor is known to exhibit good safety even upon systemic administration for a long term. However, limitations are imposed on the topical administration of the 5α-reductase inhibitor, such as ointments or injections, especially injections because irritations, such as edema, erythema, scleroderma, etc. arise locally at the application site. In the present invention, the non-steroidal anti-inflammatory drug and/or a corticosteroid, which are both highly anti-inflammatory, is introduced into a sustained-release lipid pre-concentrate to provide a pharmaceutical composition that is highly safe in spite of the local administration of the composition.

According to the present invention, when the composition is contacted with the presence of the aqueous fluid, the composition forms the liquid crystal from which the pharmaceutical active ingredient 5α-reductase inhibitor can be released in a sustained manner, which maintains the therapeutic effect for a long term to improve the patient's convenience. In addition, the 5α-reductase inhibitor in the pharmaceutical composition shows high stability for a long period of time.

The pharmaceutical composition of the present invention may be prepared by mixing a) one or more sorbitan unsaturated fatty acid esters, b) one or more phospholipids, c) one or more liquid crystal hardeners, d) one or more 5α-reductase inhibitors, and e) at least one selected from among a non-steroidal anti-inflammatory drug, and a corticosteroid at room or ordinary temperature. As needed, heat or a homogenizer may be used in the preparation of the pharmaceutical composition of the present invention. In this case, the homogenizer may be selected from among high-pressure homogenizers, ultrasonic homogenizers, and shear homogenizers.

According to the present invention, an organic solvent for solubilizing the pharmaceutically active ingredient 5α-reductase inhibitor may be further used. Preferably, the solvent may be selected from the group consisting of n-methylpyrrolidone, dimethyl sulfoxide, benzyl alcohol, benzyl benzoate, dimethylacetamide, ethanol, and a mixture thereof, but without limitations thereto.

As described above, the pharmaceutical composition of the present invention exists as the liquid phase in the absence of the aqueous fluid and forms the liquid crystals in the presence of the aqueous fluid. As it can be applied to the body using a method selected from among injection, coating, dripping, padding spraying, etc., the pharmaceutical composition of the present invention may be formulated into various dosage forms including injections, ointments, gels, lotions, liquids, suspensions, sprays, inhalants, eye drops, adhesives, patches, etc. Particularly, when an injection route is taken, the pharmaceutical composition of the present invention may be administered by subcutaneous or intramuscular injection. The administration route is adjusted by the properties of the pharmacologically active ingredients.

The dose of the pharmaceutical composition of the present invention may be the same as the well-known dose of the pharmacologically active ingredient employed, and may vary depending on various factors including the type of disease, the severity of disease, the patient's age and sex, etc. It may be administered parenterally depending on the properties of the pharmacologically active ingredient.

In accordance with another aspect thereof, the present invention provides a method of maintaining pharmaceutical efficacy through the sustained release of a pharmacologically active ingredient by administering the pharmaceutical composition of the present invention to a mammal including a human, and the use of the pharmaceutical composition for the sustained release of a pharmacologically active ingredient.

In accordance with further aspect thereof, the present invention provides a method for preventing or treating benign prostatic hyperplasia, androgenic alopecia or hypertrichosis, or aiding the treatment of post-radical prostatectomy by administering a composition to a mammal including a human, wherein the composition comprises a 5α-reductase inhibitor, and one selected from the group consisting of a non-steroidal anti-inflammatory drug (NSAID), a corticosteroid and a mixture thereof, and exists as a liquid phase in the absence of an aqueous fluid and forms a liquid crystal upon exposure to the aqueous fluid.

In the present invention, individual ingredients contained in the compositions, properties of the composition and sustained-release and therapeutic effects of the composition are described above.

In accordance with further still another aspect thereof, the present invention provides a method for preventing or treating benign prostatic hyperplasia, androgenic alopecia or hypertrichosis, or aiding treatment of post-radical prostatectomyby administering a composition to a mammal including a human, wherein the composition comprises:

a) a sorbitan unsaturated fatty acid ester having two or more —OH (hydroxyl) groups in its polar head;

b) a phospholipid;

c) a liquid crystal hardener that is free of ionizable groups and has a hydrophobic moiety of 15 to 40 carbon atoms comprising a bulky triacyl group or carbon ring structure;

d) a 5α-reductase inhibitor as a pharmaceutically active ingredient; and

e) a non-steroidal anti-inflammatory drug (NSAID), a corticosteroid, or a mixture thereof as a pharmaceutically active ingredient; and exists as a liquid phase in the absence of an aqueous fluid and forms a liquid crystal upon exposure to the aqueous fluid.

In the present invention, individual ingredients contained in the compositions, properties of the composition, and sustained-release and therapeutic effects of the composition are described above.

In accordance with further still another aspect thereof, the present invention provides the use of a composition in preventing or treating benign prostatic hyperplasia, androgenic alopecia or hypertrichosis, or as a therapeutic aid after radical prostatectomy, wherein the composition comprises a 5α-reductase inhibitor, and one selected from the group consisting of a non-steroidal anti-inflammatory drug (NSAID), a corticosteroid and a mixture thereof, and exists as a liquid phase in the absence of an aqueous fluid and forms a liquid crystal upon exposure to the aqueous fluid.

In the present invention, individual ingredients contained in the compositions, properties of the composition, and sustained-release and therapeutic effects of the composition are described above.

In accordance with further still another aspect thereof, the present invention provides the use of a composition in preventing or treating benign prostatic hyperplasia, androgenic alopecia or hypertrichosis, or as a therapeutic aid after radical prostatectomy, wherein the composition comprises:

a) a sorbitan unsaturated fatty acid ester having two or more —OH (hydroxyl) groups in its polar head;

b) a phospholipid;

c) a liquid crystal hardener that is free of ionizable groups and has a hydrophobic moiety of 15 to 40 carbon atoms comprising a bulky triacyl group or carbon ring structure;

d) a 5α-reductase inhibitor as a pharmaceutically active ingredient; and

e) a non-steroidal anti-inflammatory drug (NSAID), a corticosteroid, or a mixture thereof as a pharmaceutically active ingredient; and exists as a liquid phase in the absence of an aqueous fluid and forms a liquid crystal upon exposure to the aqueous fluid.

In the present invention, individual ingredients contained in the compositions, properties of the composition, and sustained-release and therapeutic effects of the composition are as described above.

### Advantageous Effects

Containing a non-steroidal anti-inflammatory drug and/or a corticosteroid in addition to a 5α-reductase inhibitor, as described hitherto, the pharmaceutical composition of the present invention, which is converted into a liquid crystal phase upon exposure to an aqueous fluid, is safe enough to significantly reduce irritation caused by the administration of the 5α-reductase inhibitor.

In addition, the pharmaceutical composition of the present invention, based on a sorbitan unsaturated fatty acid ester, is highly safe, and exists as a liquid phase in the absence of the aqueous fluid but rapidly changes into the liquid crystals upon exposure to the aqueous fluid within the body. Therefore, the pharmaceutically active ingredient 5α-reductase inhibitor is very stable in the pharmaceutical composition, and can be released in a sustained manner with the concomitant improvement of patient's medication compliance and long-term therapeutic efficacy. Hence, the pharmaceutical composition of the present invention is effective for the treatment of benign prostatic hyperplasia, androgenic alopecia andhypertrichosis or a therapeutic aid after radical prostatectomy which require a long term of administration.

### DESCRIPTION OF DRAWINGS

FIG. 1 illustrates phase changes of compositions of Comparative Examples 2 and 3, and Examples 4 and 7 upon exposure to the aqueous fluid.

FIG. 2 shows the in vivo drug release profile of the pharmaceutically active ingredient of the compositions of Comparative Example 1 and Example 4.

FIG. 3 shows in vivo test results of a local tolerance to evaluate in vivo safety of the pharmaceutical compositions of Comparative Examples 2, 3 and 4, and Examples 4 and 7.

FIG. 4 shows in vivo test results of a local tolerance to evaluate in vivo safety of the pharmaceutical compositions of Comparative Examples 3 and 5, and Example 4 according to the administration method of a non-steroidal anti-inflammatory drug.

### MODE FOR INVENTION

A better understanding of the present invention may be obtained through the following examples and Experimental examples which are set forth to illustrate, but are not to be construed as limiting the present invention.

The additives and excipients used in the present invention satisfied the requirements of the Korean Pharmacopoeia and were purchased from Aldrich, Lipoid, Croda, and Seppic.

### EXAMPLES 1 TO 301

### Preparation of Pharmaceutical Compositions

### EXAMPLE 1

A composition was prepared using the ingredients and contents as listed for Example 1 in Table 1.

All of the ingredients except the active ingredients dutasteride and lornoxicam, were admixed, and homogenized in a water bath of 25-75° C. using a homogenizer (PowerGen mode1125, Fisher) at 1,000-3,000 rpm for 0.5-3 hrs to give a lipid solution. This lipid solution was left at room temperature to come to thermal equilibrium at 25° C., followed by adding the pharmacologically active ingredient dutasteride and the non-steroidal anti-inflammatory drug lornoxicam of example thereto. Then, the ingredients were homogenized using a homogenizer for about 5-30 min at 1,000-3,000 rpm to afford pharmaceutical compositions in a solution phase.

### EXAMPLES 2 TO 30

The pharmaceutical compositions containing the lipid solutions and the active ingredients were prepared in the same manner as in Example 1, with the exception that ingredients and contents listed for Examples 2 to 30 in Tables 1 to 3 were used.

TABLE 1

| | Example | | | | | | | | | | | |
|---|---|---|---|---|---|---|---|---|---|---|---|---|
| (Unit: mg) | 1 | 2 | 3 | 4 | 5 | 6 | 7 | 8 | 9 | 10 | 11 | 12 |
| Dutasteride | 9 | 9 | 9 | 9 | 9 | 2.1 | 9 | 27 | | | | 9 |
| Finasteride | | | | | | | | | 11.2 | 11.2 | 60 | 60 |
| Meloxicam | | 2 | | 5 | | | | | 2.5 | | | |
| Lornoxicam | 2 | | | | | | 2 | | | | | 1 |
| Indomethacin | | | 4 | | | 2 | | | | 4 | | |
| Ketorolac | | | | | 10 | | | 15 | | | 10 | |
| Sorbitan | 37.5 | | 35 | 45 | | 75 | 50 | | | 50 | | 102 |
| monooleate | | | | | | | | | | | | |
| Sorbitan | | 40 | | | 68 | | | 113 | 48 | | 78 | |
| sesquioleate | | | | | | | | | | | | |
| Phosphatidylcholine | | 48.3 | | 67.5 | | 97.3 | 70 | | 72.5 | | 125 | |
| Phosphatidyl | 43.4 | | 45.5 | | 78.6 | | | 174 | | 45 | | 116 |
| ethanolamine | | | | | | | | | | | | |
| Tocopherol acetate | 6 | 9 | | 15 | 17.5 | 20 | 10 | | | 18 | | 15 |
| Cholesterol | 10 | | 6.3 | 7.5 | | 13 | 7.5 | 10 | 5 | | 22.6 | 12 |
| Ubiquinone | 2 | | | | | | | | 6.8 | | | |
| Benzyl benzoate | | | 5 | | 5 | | | 10 | | 10 | | |
| Ethanol | | 10 | | 20 | | 22.7 | 15 | 25 | | 5 | 35 | |
| NMP | | | 15 | 30 | | 12.5 | 20 | | | | | 7.5 |
| DMSO | | 10 | | 20 | 12.5 | | 15 | 60 | 15 | | | |

| | |
|---|---|
| Form in aqueous | formation of the liquid crystal |
| phase | |

TABLE 2

| | Example | | | | | | | | | | | |
|---|---|---|---|---|---|---|---|---|---|---|---|---|
| (Unit: mg) | 13 | 14 | 15 | 16 | 17 | 18 | 19 | 20 | 21 | 22 | 23 | 24 |
| dutasteride | 9 | 9 | 9 | 2.1 | 9 | 27 | | 2.1 | | | | 9 |
| finasteride | | | 11.2 | | | | 11.2 | 11.2 | 11.2 | 11.2 | 60 | 60 |
| Meloxicam | | 2 | | 5 | | | | | 2.5 | | | |
| Lornoxicam | 2 | | | | | | | | | 4 | 2 | 6 |
| Indomethacin | | | 4 | | | | | | 2 | | | |
| Ketorolac | | | | | | | | | | | 15 | |
| Betamethasone | | | | | 2 | | | 4 | | | | 4 |
| Dexamethasone | | | | | | 4 | 2 | | | 4 | | |
| Sorbitan | | 42.5 | | 37.5 | | 85 | | | 50 | 47.5 | | 85 |
| monooleate | | | | | | | | | | | | |
| Sorbitan | 45 | | 60 | | 45.5 | | 65.5 | 75 | | | 125 | |
| sesquioleate | | | | | | | | | | | | |
| Phosphatidyl- | 50 | | 77.5 | | 60 | 115 | 82.5 | 86.5 | | 65 | | 120 |
| choline | | | | | | | | | | | | |
| Phosphatidyl | | 55 | | 45.5 | | | | | 67.5 | | 170 | |
| ethanolamine | | | | | | | | | | | | |
| Tocopherol | 10 | | 12.5 | 15 | | 30 | 20 | 17.5 | 10 | | 5 | |
| acetate | | | | | | | | | | | | |
| cholesterol | | 7.5 | 10 | 7.5 | 5 | | 15 | 5 | | | 10 | 10 |
| ubiquinone | 2 | | | | | | | | | 2 | | 1 |
| benzyl benzoate | | 5 | 5 | | 5 | | | 5 | | 5 | 5 | 2.5 |
| Ethanol | 5 | 10 | 10 | 12.5 | | 25 | 25 | 5 | 10 | 15 | 25 | 10 |
| NMP | | | 15 | | 15 | 30 | | 20 | 10 | | 20 | 20 |
| DMSO | | 10 | | 20 | | 20 | 10 | 10 | | 10 | | |

| | |
|---|---|
| Form in aqueous | formation of the liquid crystal |
| phase | |

TABLE 3

| | Example | | | | | |
|---|---|---|---|---|---|---|
| (Unit: mg) | 25 | 26 | 27 | 28 | 29 | 30 |
| dutasteride | 9 | 9 | 9 | 2.1 | 9 | 27 |
| finasteride | | | 11.2 | | | |
| meloxicam | | 2 | | 5 | | |
| lornoxicam | 2 | | | | 4 | 4 |
| indomethacin | | | 4 | | | |
| Ketorolac | 5 | | | | 10 | |
| Betamethasone | | 4 | | 2 | | |
| Dexamethasone | | | 4 | | | 2 |
| Sorbitan monooleate | | 45.5 | | 55.5 | | 92.5 |
| Sorbitan sesquioleate | 55 | | 65 | | 52.5 | |
| phosphatidylcholine | | | 82.5 | 62.5 | 67.5 | 110 |
| Phosphatidyl ethanolamine | 60 | 52.5 | | | | |
| Tocopherol acetate | 5 | | 15 | 5 | | 25 |
| cholesterol | 5 | 7.5 | | 10 | 7.5 | |
| ubiquinone | | | | | | 1 |
| benzyl benzoate | | 5 | 10 | | 5 | |
| Ethanol | 15 | 10 | 20 | 20 | | 20 |
| NMP | 10 | | | 15 | 20 | 25 |
| DMSO | | 15 | | | 5 | 20 |

| | |
|---|---|
| Form in aqueous | formation of the liquid crystal |
| phase | |

### COMPARATIVE EXAMPLES 1 TO 4

### COMPRATIVE EXAMPLE 1

In Comparative Example 1, Avodart soft capsule 0.5 mg containing dutasteride as a pharmaceutically active substance was used.

### COMPARATIVE EXAMPLE 2

A lipid solution was prepared using the ingredients and contents as listed for Comparative Example 2 in Table 4.

The ingredients were homogeneously mixed in a water bath of 25˜75° C., using a homogenizer (PowerGen model 125, Fisher) for about 0.5˜3 hrs at 1,000-3000 rpm to give a lipid solution.

### COMPARATIVE EXAMPLES 3 AND 4

The pharmaceutical compositions containing the lipid solutions and the pharmaceutically active ingredients were prepared in the same manner as in Example 1, with the exception that ingredients and contents listed for Comparative Examples 3 and 4 in Table 4 were employed.

TABLE 4

| | Comparative | | |
|---|---|---|---|
| | Example | | |
| (unit: mg) | 2 | 3 | 4 |
| Dutasteride | | 9 | 9 |
| Finasteride | | | |
| Chlorpheniramine | | | |
| Diphenhydramine | | | 7 |
| Sorbitan monooleate | 45 | 52.3 | 62.1. |
| Sorbitan sesquioleate | | | |
| Phosphatidyl choline | 67.5 | 72.7 | 79.2 |
| Phosphatidyl ethanolamine | | | |
| Tocopherol acetate | 15 | 12.5 | 15 |
| Cholesterol | 7.5 | 10 | 5 |
| Benzyl benzoate | | | |
| Ethanol | 20 | 20 | 20 |
| NMP | | 30 | 25 |
| DMSO | | 20 | 17.3 |

| | |
|---|---|
| Form in aqueous phase | formation of |
| | liquid crystal |

### COMPARATIVE EXAMPLE 5

A composition of Comparative Example 5 was prepared using ingredients and contents listed for Comparative Example 5 in Table 5. A solution of Meglumine and Poloxamer 188 solved in water was mixed with a solution of meloxicam and glycin solved in ethanol and glycofurol at room temperature by stirring to give a meloxicam suspension.

TABLE 5

| (Unit: mg) | Comparative Example 5 |
|---|---|
| Meloxicam | 5 |
| Ethanol | 75 |
| Glycofurol | 50 |
| Poloxamer 188 | 25 |
| Glycine | 3 |
| Meglumine | 1.5 |
| Water | 340 |

### EXPERIMENTAL EXAMPLE 1

### Contents of Pharmacological Active Ingredients in Pharmaceutical Compositions

The contents of pharmacologically active substances were confirmed in the pharmaceutical compositions prepared in Examples. In this regard, the content of the pharmaceutically active substance dutasteride was examined and the results was showed by Table 6. The content of dutasteride is quantitated by HPLC under the following analysis condition.

<HPLC analysis condition for dutasteride>

Column: 4.6×250 mm, 5 μm

Column Temperature: 35° C.

Detector: UV absorption spectrometer (wavelength: 220 nm)

Flow rate: 1.0 mL/min

Injection load: 10 μL

Mobile phase: a mixture of pure water, acetonitrile and trifluoroacetic acid (volume ratio 48:52:0.025)

TABLE 6

| | Comparative Example | | Example | | | |
|---|---|---|---|---|---|---|
| | 3 | 4 | 4 | 6 | 7 | 18 |
| Content (Unit: %) | 98.7 | 101.2 | 99.2 | 102.4 | 101.7 | 100.6 |

As can be seen in Table 6, the pharmaceutical compositions of Comparative Examples 3 and 4 and Examples 4, 6, 7 and 18 were all measured to contain ideal levels of dutasteride, which were within standard content (100%)±3%.

### EXPERIMENTAL EXAMPLE 2

### Formation of Liquid Crystal in Aqueous Fluid

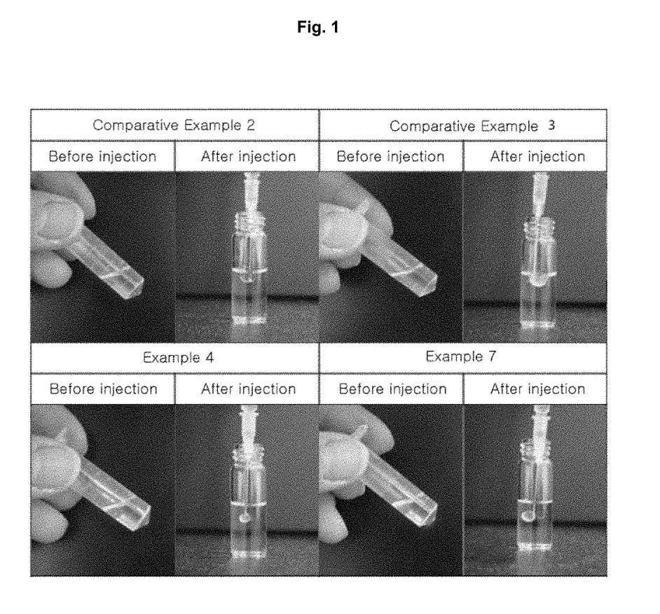

It is confirmed whether the pharmaceutical compositions prepared in Examples form ideal liquid crystals in an aqueous fluid. In this regard, the compositions of Comparative Examples 2 to 4, and Examples 1 to 30, which were all in liquid states, were loaded into syringes and then applied to 2 g of PBS (pH 7.4). Whether or not the compositions of Examples 1 to 30 converted into the liquid crystals is given in Tables 1 to 3 while results from compositions of Examples 4 and 7 and Comparative Examples 2 and 3 are depicted in FIG. 1.

The pharmaceutical compositions prepared in Examples 4 and 7 and Comparative Examples 2 and 3 existed in the liquid phase in the absence of the aqueous fluid. When introduced into the aqueous fluid (PBS), these pharmaceutical compositions in the liquid phase of Examples 4 and 7 were converted into the liquid crystals, indicating that the pharmaceutical compositions can form the liquid crystals even though they contain the pharmacologically active ingredients, that is, the 5α-reductase inhibitor, the non-steroidal anti-inflammatory drug and the corticosteroid.

### EXPERIMENTAL EXAMPLE 3

### In Vivo PK Profile of Pharmaceutical Compositions

Drug release behaviors in vivo from the compositions of the present invention were examined in the following test. Using a disposable syringe, the composition of Example 4 was subcutaneously injected at a dutasteride dose of 30.0 mg/kg (corresponding to a 30-day dose for humans) into the back of 6 SD rats (male), 9 weeks old with an average body weight of 300 g.

For comparison with PK profiles of an oral formulation, the composition of Comparative Example 1 was orally administered at a dutasteride dose of 1.7 mg/kg (corresponding to a 1-day dose for humans) into 6 SD rats (male), 9 weeks old with an average body weight of 300 g.

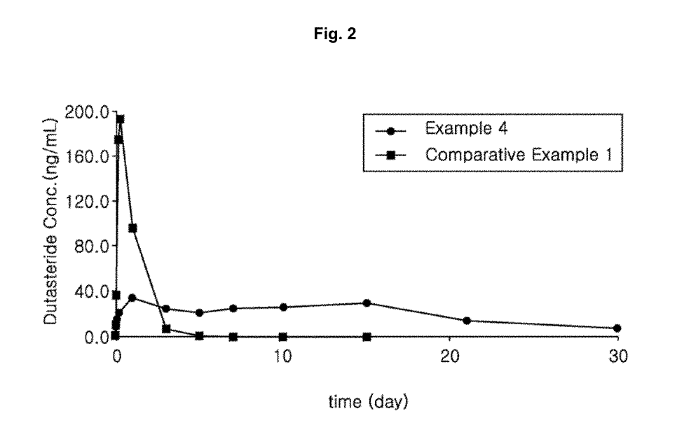

Dutasteride concentrations in plasma samples taken from the SD rats were monitored for 30 days using LC-MS/MS (liquid chromatography-mass spectrometry) to obtain PK profiles (pharmacokinetic profiles). The means of measurements taken of the 6 rats used in each experiment are plotted in FIG. 2

When orally administered at a 1-day dose, the composition of Comparative Example 1 released the drug at an initial concentration of about 192 ng/mL whereas the composition of Example 4 had an initial concentration of about 36 ng/mL even though administered once at a 1-month dose. From these results, it is confirmed that the composition of Example 1 achieves a remarkable improvement in an initial burst of a typical problem with the oral formulations. In addition, the composition of Example 4 maintained almost constant effective plasma levels of the drug over 30 days. The data obtained above demonstrate that the composition of Example 4 as a sustained-release formulation exhibited ideal pK behavior and excellent sustained release compared to Comparative Example 1.

### EXPERIMENTAL EXAMPLE 4

### Assay for In vivo Local Tolerance

The composition of the present invention was assayed for in vivo safety as follows.

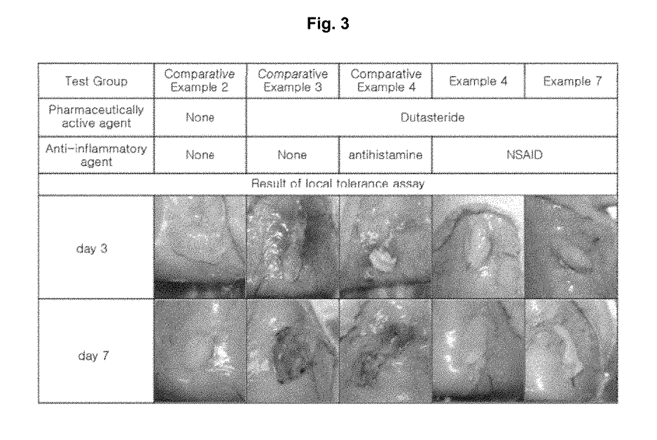

Using a disposable syringe, each of the compositions of Comparative Examples 3 and 4, which contained the 5α-reductase inhibitor dutasteride alone and in combination with the anti-inflammatory antihistamine agent, respectively, was subcutaneously injected at a dutasteride dose of 30.0 mg/kg (corresponding to a 30-day dose for humans) into the back of 6 SD rats (male), 9 weeks old with an average body weight of 300 g. As can be seen in FIG. 3, local irritations such as edema, erythema, sclerodema, etc. were observed at the administration site. Comparative Example 4 further employing the antihistamine agents exhibited alleviated local irritations at day 3 compared to Comparative Example 3, but similar irritations were observed at day 7.

In order to examine whether the irritations of the compositions of Comparative Examples 3 or 4 were caused by the active ingredient or the lipid solution, the composition of Comparative Example 2, which contained no pharmaceutically active ingredients, was subcutaneously injected into the back of 6 SD rats (male), 9 weeks old with an average body weight of 300 g, using a disposable syringe. The injection dose of the composition of Comparative Example 2 was identical to the total dose of the composition of Comparative Example 4. As shown in FIG. 3, the lipid solution of Comparative Example 2 did not irritate the tissue at all.

The pharmaceutical compositions of Examples 4 and 7, which contained the non-steroidal anti-inflammatory drugs meloxicam and lornoxicam, respectively, were subcutaneously injected, and local tolerance was observed at each of the injection sites. Using a disposable syringe, each composition was injected at a dutasteride dose of 30.0 mg/kg (corresponding to a 30-day dose for humans) into the back of 6 SD rats (male), 9 weeks old with an average body weight of 300 g.

Like the composition of Comparative Example 2 that contained no pharmaceutically active substances, the compositions containing the non-steroidal anti-inflammatory drug meloxicam (Example 4) and lornoxicam (Example 7) exhibited no irritations, demonstrating its excellent safety. Further, the compositions of other Examples had reduced local irritations. Accordingly, the pharmaceutical compositions comprising the non-steroidal anti-inflammatory drug and/or a corticosteroid were found to reduce the irritation caused by the topical administration of the 5α-reductase inhibitor.

### EXPERIMENTAL EXAMPLE 5

### Assay for In Vivo Local Tolerance According to Administration Method and Route of Anti-inflammatory Drug

The pharmaceutical compositions were in vivo assayed for hypodermic safety according to administration methods and routes of the non-steroidal anti-inflammatory drugs.

Groups that were administered, respectively, with the composition of Comparative Example 3 which contained the pharmaceutically active substance dutasteride alone, with the composition of Comparative Example 3 plus oral administration of the composition of Comparative Example 5, and with the composition of Comparative Example 3 plus subcutaneous injection of the composition of Comparative Example 5 were observed for anti-inflammation effects. Also, the composition of Example 4 which contained dutasteride and the non-steroidal anti-inflammatory drug meloxicam was also examined for the anti-inflammation effects.

Each of the compositions of Comparative Example 3 and Example 4 was subcutaneously injected at a dutasteride dose of 30.0 mg/kg (corresponding to a 30-day dose for humans) into the back of 6 SD rats (male), 9 weeks old with an average body weight of 300 g. Meanwhile, the composition of Comparative Example 5 was orally or subcutaneously administered at a meloxicam dose of 17.0 mg/kg to 6 SD rats (male), 9 weeks old with an average body weight of 300 g.

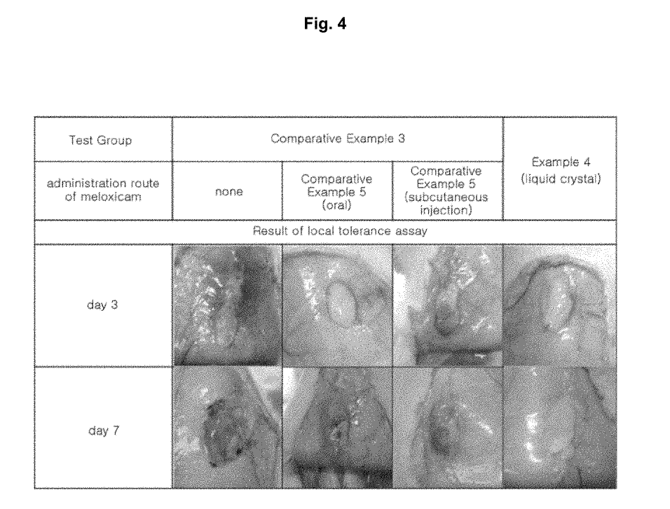

As shown in FIG. 4, the group administered with the non-steroidal anti-inflammatory drug-free composition of Comparative Example 3 alone underwent local irritations, such as edema, erythema, sclerodema, etc., at the injection site. Likewise, similar irritations were also observed in the group to which the composition of Comparative Example 3 was introduced, together with a meloxicam suspension, by subcutaneous injection, respectively. The group to which the composition of Comparative Example 3 and a meloxicam suspension were introduced through subcutaneous oral routes, respectively, was observed to have alleviated the local irritations on day 3, but to be similar in the local irritation to the group administered with the composition of Comparative Example 3 alone on day 7. Hence, it was confirmed that the oral administration of the anti-inflammatory agent elicited an anti-inflammatory effect only in the early stage. In contrast, the group subcutaneously injected with the composition of Example 4 comprising both dutasteride and meloxicam did not suffer from the local irritations for 7 days. Compositions containing corticosteroid and/or non-steroidal anti-inflammatory drugs other than meloxicam were assayed for the local tolerance according to administration methods and routes, and similar results were obtained. Taken together, the data obtained above indicate that effects of the anti-inflammatory drugs are different depending on the administration method and route, and are improved and maintained for a prolonged period of time when the anti-inflammatory drugs are introduced into the liquid crystal in vivo, thus guaranteeing excellent safety to the composition.

1. A composition, comprising:
a 5α-reductase inhibitor; and
a non-steroidal anti-inflammatory drug (NSAID), corticosteroid, or a combination thereof,
wherein the composition exists as a lipid phase in the absence of an aqueous fluid and forms a liquid crystal upon exposure to the aqueous liquid.

2. The composition of claim 1, wherein the 5α-reductase inhibitor is selected from the group consisting of dutasteride, finasteride, bexlosteride, epristeride, izonsteride, lapisteride, turosteride, a pharmaceutically acceptable salt thereof, and a combination thereof.

3. The composition of claim 1, wherein the 5α-reductase inhibitor is selected from the group consisting of dutasteride, finasteride, a pharmaceutically acceptable salt thereof, and a combination thereof.

4. The composition of claim 1, wherein the 5α-reductase inhibitor is dutasteride or a pharmaceutically acceptable salt thereof.

5. The composition of claim 1, wherein the non-steroidal anti-inflammatory drug is selected from the group consisting of aceclofenac, acemetacin, alminoprofen, amfenac, apazone, aspirin, bromfenac, bufexamac, celecoxib, choline salicylate, cinnoxicam, clonixin, dexibuprofen, dexketoprofen, diclofenac, diflunisal, emorfazone, etodolac, etoricoxib, ethenzamide, felbinac, fenoprofen, flufenamic acid, flurbiprofen, ibuprofen, imidazole salicylate, indomethacin, isopropylantipyrine, ketoprofen, ketorolac, lornoxicam, loxoprofen, meclofenamate, meloxicam, mefenamic acid, morniflumate, nabumetone, naproxen, nefopam, nimesulide, oxaprozin, oxyphenbutazone, pelubiprofen, phenylbutazone, piroxicam, pranoprofen, proglumetacin, rofecoxib, salsalate, salicylate, sulindac, talniflumate, tenoxicam, tiaprofenic acid, tolfenamic acid, tolmetin, valdecoxib, zaltoprofen, a pharmaceutically acceptable salt thereof, and a combination thereof.

6. The composition of claim 1, wherein the non-steroidal anti-inflammatory drug is selected form the group consisting of dexketoprofen, ibuprofen, indomethacin, ketoprofen, ketorolac, lornoxicam, meloxicam, nefopam, piroxicam, a pharmaceutically acceptable salt thereof, and a combination thereof.

7. The composition of claim 1, wherein the non-steroidal anti-inflammatory drug is selected from the group consisting of lornoxicam, meloxicam, a pharmaceutically acceptable salt thereof, and a combination thereof.

8. The composition of claim 1, wherein the corticosteroid is selected from the group consisting of beclomethasone dipropionate, betamethasone, budesonide, deflazacort, dexamethasone, difluprednate, epinephrine, fludrocortisone, fluocinolone acetonide, fluocortin, fluorometholone, fluticasone, hydrocortisone, methylprednisolone, prednisolone, prednisone, triamcinolone, a pharmaceutically acceptable salt thereof, and a combination thereof.

9. The composition of claim 1, wherein the corticosteroid is selected from the group consisting of betamethasone, dexamethasone, epinephrine, hydrocortisone, methylprednisolone, prednisolone, triamcinolone, a pharmaceutically acceptable salt thereof, and a combination thereof.

10. The composition of claim 1, wherein the corticosteroid is selected from the group consisting of betamethasone, dexamethasone, a pharmaceutically acceptable salt thereof, and a combination thereof.

11. The composition of claim 1, wherein the composition is a formulation for an oral administration.

12. The composition of claim 1, wherein the composition is a formulation for injection.

13. The composition of claim 1, wherein the composition is used for treating benign prostatic hyperplasia, androgenic alopecia, or hypertrichosis, or as a therapeutic aid after radical prostatectomy.

14. A composition, comprising:
a) a sorbitan unsaturated fatty acid ester having two or more —OH (hydroxyl) groups in its polar head;
b) a phospholipid;
c) a liquid crystal hardener that is free of ionizable groups and has a hydrophobic moiety of 15 to 40 carbon atoms comprising a bulky triacyl group or carbon ring structure;
d) a 5α-reductase inhibitor as a pharmaceutically active substance; and
e) a non-steroidal anti-inflammatory drug (NSAID), a corticosteroid, or a mixture thereof,
wherein the composition exists as a liquid phase in the absence of an aqueous fluid, and forms a liquid crystal upon exposure to the aqueous fluid.

15. The composition of claim 14, wherein the sorbitan unsaturated fatty acid ester is selected from the group consisting of sorbitan monooleate, sorbitan monolinoleate, sorbitan monopalmitoleate, sorbitan monomyristoleate, sorbitan sesquioleate, sorbitan sesquilinoleate, sorbitan sesquipalmitoleate, sorbitan sesquimyristoleate, sorbitan dioleate, sorbitan dilinoleate, sorbitan dipalmitoleate, sorbitan dimyristoleate, and a combination thereof.

16. The composition of claim 14, wherein the sorbitan unsaturated fatty acid ester is selected from the group consisting of sorbitan monooleate, sorbitan sesquioleate, sorbitan monolinoleate, sorbitan monopalmitoleate, sorbitan monomyristoleate, and a combination thereof.

17. The composition of claim 14, wherein the phospholipid contains a saturated or unsaturated alkyl ester group of 4 to 30 carbon atoms, and is selected from the group consisting of phosphatidylcholine, phosphatidylethanolamine, phosphatidylserine, phosphatidylglycerine, phosphatidylinositol, phosphatidic acid, sphingomyelin, and a combination thereof.

18. The composition of claim 14, wherein the liquid crystal hardener is selected from the group consisting of triglyceride, retinyl palmitate, tocopherol acetate, cholesterol, benzyl benzoate, ubiquinone, and a combination thereof.

19. The composition of claim 14, wherein the liquid crystal hardener is selected from the group consisting of tocopherol acetate, cholesterol, benzyl benzoate, and a combination thereof.

20. The composition of claim 1 or 14, further comprising an organic solvent.

21. (canceled)
